SEQUENCE LISTING (10 sequences)

SEQ ID NO: 1 - DNA, 699 bp, Homo sapiens
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 2 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 157 aa, Homo sapiens [sequence omitted]

SEQ ID NO: 5 - DNA, 1365 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcaga

SEQ ID NO: 6 - DNA, 2258 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaaaa

SEQ ID NO: 7 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 8 - DNA, 1383 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagt

SEQ ID NO: 9 - DNA, 3682 bp, Homo sapiens
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 10 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

(19) United States
(12) Patent Application Publication
(10) Pub. No.: US 2017/0165361 A1
(43) Pub. Date: Jun. 15, 2017

(54) TREATMENT OF LYSOSOMAL DISORDERS (ANTI-TNF THERAPY)

(71) Applicant: Oxford University Innovation Limited, Oxford (GB)

(72) Inventors: Frances PLATT, Oxford (GB); Nick PLATT, Oxford (GB); Raashid LUQMANI, Oxford (GB)

(21) Appl. No.: 15/325,354

(22) PCT Filed: Jul. 8, 2015
(86) PCT No.: PCT/GB2015/051980

§ 371 Date: Jan. 10, 2017

(30) Foreign Application Priority Data
Jul. 11, 2014 (GB) 1412411.9

(51) Int. Cl. A61K 39/395; A61K 31/7052; C07K 16/24; A61K 38/19

(52) U.S. Cl. CPC A61K 39/3955; A61K 38/191; A61K 31/7052; C07K 16/241; A61K 2039/55522

(57) ABSTRACT

The invention relates to methods for treating a Lysosomal storage disease (LSD) in a patient. Kits for use in such methods are also provided.

### FIELD OF THE INVENTION

The invention relates to methods for preventing or treating a lysosomal storage disorder (LSD) in a patient. The invention also provides Tumour Necrosis Factor (TNF) antagonists and kits for preventing and treating a LSD. The inventors have surprisingly shown that TNF antagonists can be used for preventing or treating a LSD.

### BACKGROUND OF THE INVENTION

Lysosomal storage disorders (LSDs) are a group of inherited metabolic diseases caused by defects in lysosomal homeostasis. To date, LSDs encompass over 60 diseases, with a collective clinical frequency of 1:5000 live births. These diseases can be classified into two main groups: primary storage disorders resulting from a direct deficiency in degradation pathways (typically lysosomal enzyme deficiency disorders); and secondary storage disorders which are caused by malfunctioning downstream lysosomal proteins. In most cases, multiple organs and tissues are involved. Region-specific neurodegeneration and chronic neuroinflammation are featured in the majority of these diseases.

The pathology of LSDs affects many of the body's systems, but most commonly the nervous system. Mental retardation is a common symptom. Such disorders are generally severely progressive and unremitting. They tend to present in the first few years of life and the severe progression results in frequent hospitalization. If left untreated, patients often die in their mid-teens.

There is very limited knowledge as to the profile of pro-inflammatory molecules in LSDs.

Current therapeutic approaches for LSDs are limited. There are few, if any, curative treatments and many of the therapeutic options merely improve quality of life. Some LSDs have been responsive to bone marrow transplantation or enzyme replacement therapy. Some benefit has also been reported in a clinical trial using an inhibitor of glycosphingolipid (GSL) biosynthesis: the imino sugar drug, miglustat (Patterson et al., Rev Neurol (separata) 2006; 43: 8). However, there are currently no non-specific treatments that benefit all LSDs.

There is therefore a need to develop improved treatments of LSDs.

### SUMMARY OF THE INVENTION

The inventors have surprisingly shown that Tumour Necrosis Factor (TNF) antagonists can be used for preventing or treating a lysosomal storage disorder (LSD) in a patient.

The invention provides a method for preventing or treating a LSD in a patient, wherein the method comprises administering to the patient a TNF antagonist and thereby treating the LSD.

The invention further provides a TNF antagonist for use in a method for preventing or treating a LSD.

The invention further provides an agent for the prevention or treatment of a LSD, comprising a TNF antagonist as an active ingredient.

The invention further provides a kit for preventing or treating a LSD, comprising a means for diagnosing or prognosing a LSD and a TNF antagonist.

### BRIEF DESCRIPTION OF THE FIGURES

FIG. 1 shows biologics targeting TNF in clinical use and a simplified structural representation of anti-TNF biologics.

FIG. 2 shows effect of TNF-targeting biologics on survival of Niemann-Pick type C disease (Npc1^{-/-}) mice. (A) Kaplan-Meier survival curve (percent survival) and (B) Average survival ages. Data presented as mean±SEM, *p<0.05 calculated using one-way ANOVA with Tukey's post hoc test (n=5-14). These biologics do not prolong the life span of NPC1 mice.

FIG. 3 shows effect of TNF-targeting biologics on body weight loss of Npc1^{-/-} mice. Data presented as mean (percent of initial body weight)±SEM. *p<0.05 and **p<0.01 calculated using one-way ANOVA with Tukey's post hoc test (n=5-14). * denotes comparison vs. controls s.c., denotes comparison vs. untreated controls, + denotes comparison vs. Enbrel and # denotes comparison vs. Humira.

FIG. 4 shows beneficial effects of TNF-targeting biologics on neurological function as measured by (A) rearing and (B) centre rearing of Npc1^{-/-} mice. Data presented as mean (rearing count)±SEM. *p<0.05 and **p<0.01 calculated using one-way ANOVA with Tukey's post hoc test (n=5-14).

FIG. 5 shows effect of TNF-targeting biologics on fine tremor of Npc1^{-/-} mice. Tremor amplitudes quantified as averaged fine tremor amplitude (over the range 31-51 Hz). Data presented as mean±SEM. *p<0.05 and ***p<0.001 calculated using one-way ANOVA with Tukey's post hoc test (n=5-14).

FIG. 6 shows beneficial effects of biologic treatment on locomotor activity. The AmLogger setup produces various activity parameters: activity count (A), rearing count (B), time spent being mobile (C), active (D) and rearing (E). Occasions where the animals moved from one end of the box to the other are also indicated (Front-to-rear movement; F). Enbrel®-treated animals displayed higher activity levels, including a significantly longer time spent on rearing at 8 weeks (F) and time being active at 9 weeks (C) (n=7).

FIG. 7 shows beneficial effects of biologic treatment on single paw gait parameters. Trends that are consistent in all four paws are represented by arrows, where their implicated beneficial or worsening effects are indicated in green and red respectively. The first column (NPC) indicates parameters that are affected in NPC disease progression while the second (NPC+Enbrel®) shows the effects following Enbrel® treatment in Npc1^{-/-} mice.

FIG. 8 shows the chemical structures of LysoTracker®. (A) is LysoTracker® blue. (B) is LysoTracker® green. (C) is LysoTracker® red.

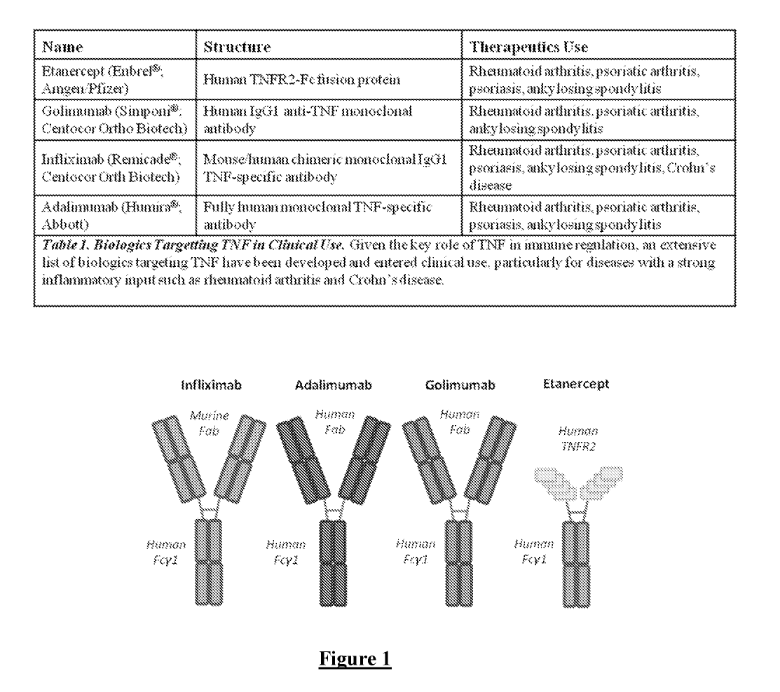

### BRIEF DESCRIPTION OF THE SEQUENCES

SEQ ID NO: 1 shows the sequence of TNF cDNA, Genbank-NM_000594.3
SEQ ID NO: 2 shows the sequence of TNF RNA, Genbank-NM_000594.3
SEQ ID NO: 3 shows the sequence of TNF protein membrane form, Genbank-NP_000585.2
SEQ ID NO: 4 shows the sequence of TNF protein soluble form, Genbank-NP_000585.2, which corresponds to amino acids 77-233 of SEQ ID NO:3.
SEQ ID NO: 5 shows the sequence of TNFR 1 cDNA, Genbank-NM_001065.3
SEQ ID NO: 6 shows the sequence of TNFR 1 RNA, Genbank-NM_001065.3
SEQ ID NO: 7 shows the sequence of TNFR 1 protein, Genbank-NP_001056.1
SEQ ID NO: 8 shows the sequence of TNFR 2 cDNA, Genbank-NM_001066.2
SEQ ID NO: 9 shows the sequence of TNFR 2 RNA, Genbank-NM_001066.2
SEQ ID NO: 10 shows the sequence of TNFR 2 protein, Genbank-NP_001057.1

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “an antagonist” includes “antagonists”, reference to “an antibody” includes two or more such antibodies, reference to “a LSD” includes two or more such LSDs, and the like.

All publications, patents and patent applications cited herein, whether supra or infra, are hereby incorporated by reference in their entirety.

### Method of the Invention

The present invention relates to a method for treating a LSD in a patient. The method for treating the LSD comprises administering to the patient a TNF antagonist. The invention also concerns a TNF antagonist for use in a method for preventing or treating a LSD in a patient. The invention also concerns use of a TNF antagonist in the manufacture of a medicament for treating or preventing a LSD in a patient.

The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human.

### Lysosomal Storage Disorders (LSDs)

A LSD is any disorder that involves dysfunction or disruption in the late endosomal/lysosomal system.

A LSD is any disorder that involves an increased volume and/or pH of the endosomal/lysosomal system. The LSD may involve increased storage of lipid or non-lipids. The LSD may be a primary lysosomal hydrolase defect, a post-translational processing defect of lysosomal enzymes, a trafficking defect for lysosomal enzymes, a defect in lysosomal enzyme protection, a defect in soluble non-enzymatic lysosomal proteins, a transmembrane (non-enzyme) protein defect or an unclassified defect.

Primary lysosomal hydrolase defects include, but are not limited to, Gaucher disease (Glucosylceramidase defect), GM1 gangliosidosis (GM1-β-galactosidase defect), Tay-Sachs disease (β-Hexosaminidase A defect), Sandhoff disease (β-Hexosaminidase A+B defect), Fabry disease (α-Galactosidase A defect), Krabbe disease (β-Galactosyl ceramidase defect), Niemann-Pick disease Type A and B (Sphingomyelinase defect), Metachromatic leukodystrophy (Arylsulphatase A defect), MPS IH (Hurler syndrome; α-Iduronidase defect), MPS IS (Scheie syndrome; α-Iduronidase defect), MPS II (Hunter syndrome; Iduronate sulphatase defect), MPS IIIA (Sanfilippo A syndrome; Heparan sulphamidase defect), MPS IIIB (Sanfilippo B syndrome; Acetyl α-glucosaminidase defect), MPS IIIC (Sanfilippo C syndrome; Acetyl CoA: α-glucosaminide N-acetyltransferase defect), MPS IIID (Sanfilippo D syndrome; N-Acetyl glucosamine-6-sulphatase defect), MPS IV A (Morquio A disease; Acetyl galactosamine-6-sulphatase defect), MPS IVB (Morquio B disease; β-Galactosidase defect), MPS V (redesignated MPS IS), MPS VI (Maroteaux Lamy Syndrome; Acetyl galactosamine-4-sulphatase (Arylsulphatase B) defect), MPS VII (Sly Syndrome; β-Glucuronidase defect), MPS IX (Hyaluronidase defect), Wolman disease (WD; Acid lipase defect), Farber disease (Acid ceramidase defect), Cholesteryl ester storage disease (Acid lipase defect), Pompe disease (Type II; α 1,4-Glucosidase defect), Aspartylglucosaminuria (Glycosylasparaginase defect), Fucosidosis (α-Fucosidase defect), α-Mannosidosis (α-Mannosidase defect), β-Mannosidosis ((3-Mannosidase defect), Schindler disease (N-Acetylgalactosaminidase defect), Sialidosis (α-Neuraminidase defect), Infantile neuronal ceroid lipofuscinoses (CLN1; Palmitoyl protein thioesterase defect) and Late infantile neuronal ceroid lipofuscinoses (CLN2; Carboxypeptidase defect).

Post-translational processing defects of lysosomal enzymes include, but are not limited to, Mucosulphatidosis (MSD; Multiple sulphatase defect).

Trafficking defects for lysosomal enzymes include, but are not limited to, Mucolipidosis type II (I-cell disease; N-Acetyl glucosamine phosphoryl transferase defect), Mucolipidosis type IIIA (pseudo-Hurler polydystrophy; N-Acetyl glucosamine phosphoryl transferase defect) and Mucolipidosis type IIIC.

Defects in lysosomal enzyme protection include, but are not limited to, Galactosialidosis (Protective protein cathepsin A (PPCA) defect), β-galactosidase defects and neuraminidase defects.

Defects in soluble non-enzymatic lysosomal proteins include, but are not limited to, GM2 activator protein deficiency (Variant AB), Sphingolipid activator protein (SAP) deficiency and Neuronal ceroid lipofuscinoses (NCL) (CLN5).

Transmembrane (non-enzyme) protein defects include, but are not limited to, Danon disease (Lysosome-associated membrane protein 2 (LAMP2) defect), Niemann-Pick Type C (NPC1 and NPC2 defect), Cystinosis (Cystinosin defect), Infantile free sialic acid storage disease (ISSD; Sialin defect), Salla disease (free sialic acid storage; Sialin defect), Juvenile neuronal ceroid lipofuscinoses (CLN3, Batten disease), Neuronal ceroid lipofuscinoses (NCL) (CLN6 and CLN8) and Mucolipidosis type IV (Mucolipin defect).

Unclassified defects include, but are not limited to, Neuronal ceroid lipofuscinoses (NCL) (CLN4 and CLN7).

The ability of lysosomes to fuse with late endosomes relies upon calcium release, specifically from the late endosomal/lysosomal compartment itself. When insufficient calcium is released from acidic stores there is a complete block in late endosome-lysosome fusion. A severe human disease results from calcium deficiency in the acidic compartment, the LSD termed Niemann-Pick disease type C (NPC).

Niemann-Pick diseases are a heterogeneous group of autosomal recessive LDSs. Common cellular features include abnormal sphingomyelin (SM) storage in mononuclear phagocytic cells and parenchymal tissues, as well as (hepato)splenomegaly. Among the three main subgroups (A-C), Niemann-Pick disease type C (previously classified as NPC and NPD and now appreciated to be a single disease) is classified as a fatal neurovisceral LSD caused by abnormal intracellular cholesterol transport-induced accumulation of unesterified cholesterol in late endosome/lysosomal compartments. Clinical manifestations of NPC differ according to age of onset, where typical clinical signs include chronic jaundice in neonates, ataxia, dystonia, vertical supranuclear gaze palsy, dementia and other motor and neurological problems. A previous clinical study has indicated a mean age at diagnosis to be 10.4 years whilst the average age of death is 16.2 years old. Rarer adult-onset forms have also been reported and are probably under-diagnosed.

Outside the CNS, the cellular characteristics of NPC include abnormal accumulation of unesterified cholesterol and other lipids (e.g. GSLs) within late endosome/lysosomal compartments. Conversely, there is no net elevation in cholesterol in the CNS (although it does have an altered distribution) but there are highly elevated levels of GSLs. Progressive neurodegeneration is particularly characterized by sequential degeneration of GABAergic Purkinje neurons in the cerebellum, which parallels the onset and progression of cerebellar ataxia and other aspects of neurological dysfunctions seen during the course of NPC.

Genetic studies have shown that NPC disease is caused by mutations in either the Npc1 or Npc2 genes.

NPC is unusual as it is caused by mutations in two genes, NPC1 or NPC2, that function as part of the same cellular pathway. However, the precise mechanistic link between these two genes remains unknown and the functional roles of these proteins remains enigmatic. NPC1 encodes a multimembrane spanning protein of the limiting membrane of the late endosome/lysosome where as NPC2 is a soluble cholesterol binding protein of the lysosome.

When NPC1 is inactivated, sphingosine is the first lipid to be stored, suggesting that NPC1 plays a role in the transport of sphingosine from the lysosome, where it is normally generated as part of sphingolipid catabolism. Elevated sphingosine in turn causes a defect in calcium entry into acidic stores resulting in greatly reduced calcium release from this compartment. This then prevents late endosome-lysosome fusion, which is a calcium dependent process, and causes the secondary accumulation of lipids (cholesterol, sphingomyelin and glycosphingolipids) that are cargos in transit through the late endocytic pathway. Other secondary consequences of inhibiting NPC1 function include defective endocytosis and failure to clear autophagic vacuoles. It has been shown that the NPC1/NPC2 cellular pathway is targeted by pathogenic mycobacteria to promote their survival in late endosomes.

The term “Niemann-Pick type C disease like cellular phenotype” as used herein, means a cellular phenotype which includes: (a) abnormal cholesterol metabolism and trafficking; (b) abnormal sphingolipid storage and trafficking; and (c) defective endocytosis. Typically, the abnormal sphingolipid storage (b) involves the majority of sphingolipids in the cell being present at abnormally elevated levels. Typically, (c) comprises defective endocytosis of substantially all biomolecules in the endocytic pathway, including lipids and biomolcules other than lipids, for instance proteins.

Any LSD with secondary inhibition of the NPC disease pathway would benefit from being treated by the method of the invention.

The LSD to be treated by the method of the invention is preferably any of Niemann-Pick type C (NPC1), NPC2, Smith-Lemli-Opitz Syndrome (SLOS), an inborn error of cholesterol synthesis, Tangier disease, Pelizaeus-Merzbacher disease, the Neuronal Ceroid Lipofuscinoses, primary glycosphingolipidoses (i.e. Gaucher, Fabry, GM1, GM2 gangliosidoses, Krabbe and MLD), Farber disease and Multiple Sulphatase Deficiency.

### Tumour Necrosis Factor (TNF)

Tumour necrosis factors (or TNFs) are a group of cytokines, which can cause cell death (apoptosis). Anti-TNF therapies have been used to treat many autoimmune diseases, including rheumatoid arthritis, juvenile rheumatoid arthritis, psoriatic arthritis, ankylosing spondylitis and Crohn's disease.

The tumour necrosis factor (TNF) family embodies 19 related proteins that bind to one or more molecules within the TNF-receptor superfamily. Members of this family play key physiological roles, specifically in survival and inflammatory signalling.

TNF (previously known as TNFα) exists in two different forms (membrane-bound or as soluble trimers) and is produced primarily by macrophages and activated microglial cells. It can also be induced in B cells, T cells and fibroblasts. TNF exerts a myriad of effects by binding to two classes of TNF receptors, p55 (TNFR 1) or p57 (TNFR 2). By doing so, it can lead to different functional outcomes, such as promoting macrophage phagocytosis, enhancement of leukocyte movement or migration from the blood vessels into the tissues by increasing the permeability of endothelial layer of blood vessels; increasing the release of adhesion molecules, production of pro-inflammatory cytokines (e.g. IL-1β and IL-6), activation of anti-apoptotic nuclear factor kappa-B (NF-κB) or alternatively triggering apoptosis by caspase activation. TNF knockout mice show gross defects in the inflammatory response, including attenuated leukocyte trafficking. At low concentrations, TNF augments the host defense system; whilst higher levels of the cytokine may lead to excess inflammation or even result in a septic shock. Conversely, the anti-inflammatory cytokine IL-10 acts primarily by inhibiting the translation and expression of TNF in activated macrophages. Therefore, it is not surprising that elevated levels of TNF are implicated in various chronic inflammatory conditions including rheumatoid arthritis (RA), multiple sclerosis (MS) and Crohn's inflammatory bowel disease.

TNF is found in membrane bound and soluble forms. The cDNA of TNF is shown in SEQ ID NO: 1. The RNA sequence of TNF is shown in SEQ ID NO: 2. The amino acid sequence of the membrane bound form of TNF is shown in SEQ ID NO: 3. The amino acid sequence of the soluble form of TNF is shown in SEQ ID NO: 4. “TNF” encompasses the membrane bound form of TNF and/or the soluble form of TNF.

TNFR is found in several isoforms. The cDNA of TNFR isoform 1 (TNFR 1) is shown in SEQ ID NO: 5. The corresponding RNA and amino acid sequences are shown in SEQ ID NOs: 6 and 7. The cDNA of TNFR isoform 2 (TNFR 2) is shown in SEQ ID NO: 8. The corresponding RNA and amino acid sequences are shown in SEQ ID NOs: 9 and 10. “TNFR” encompasses TNFR 1 and/or TNFR 2.

All isoforms and allelic variants of TNF and TNFR are encompassed by “TNF” and “TNFR”. An allelic variant of TNF or TNFR is a naturally occurring variant in the nucleic acid sequence of TNF or TNFR. The invention therefore concerns antagonising an isoform or allelic variant of TNF and/or TNFR.

The present inventors have surprisingly found that that TNF antagonists can be used for treating a LSD in a patient.

### Tumour Necrosis Factor Antagonists

TNF antagonists block the function of TNF. Blocking the function of TNF encompasses any reduction in its activity and/or expression. Blocking the function of TNF encompasses any reduction in the activity and/or expression of TNFR. Blocking the function of TNF encompasses any reduction in the activity and/or expression of TNF and TNFR. Blocking the function of TNF typically results in reduced inflammation.

The TNF antagonist preferably decreases the activity of TNF. The TNF antagonist preferably decreases the activity of TNF by from about 1 to about 100%, such as at least about 50%, at least about 60%, at least about 70%, at least about 80%, at least about 90%, at least about 95% or at least about 99%.

The TNF antagonist preferably binds to TNF. The TNF antagonist preferably specifically binds to TNF. For example, the TNF antagonist preferably binds to TNF, but not to Lymphotoxin-alpha (LT-alpha), (previously known as TNFβ), even though the two types of TNF can utilise the same receptors. Specific binding is discussed in more detail below. The TNF antagonist preferably reduces the ability of TNF to bind to TNFR. The TNF antagonist preferably prevents binding of TNF to TNFR.

The TNF antagonist preferably binds to the portion of TNF that interacts with TNFR. The TNF antagonist preferably reduces the ability of TNF to activate TNFR. The TNF antagonist preferably prevents TNF from activating TNFR.

The TNF antagonist preferably reduces the bioavailability of TNF. The TNF antagonist preferably reduces the bioavailability of TNF by from about 1 to about 100%, such as at least about 50%, at least about 60%, at least about 70%, at least about 80%, at least about 90%, at least about 95% or at least about 99%. The TNF antagonist preferably neutralises or removes extracellular TNF and/or transmembrane TNF and/or receptor bound TNF. The TNF antagonist preferably binds to soluble TNF (e.g. free floating in the blood) and/or transmembrane (membrane form) TNF (that may be present at the surface of T cells and other immune cells). The TNF antagonist preferably sequesters the TNF such that it is not available to bind to TNFR.

The TNF antagonist preferably decreases the activity of TNFR. The TNF antagonist preferably decreases the activity of TNFR by from about 1 to about 100%, such as at least about 50%, at least about 60%, at least about 70%, at least about 80%, at least about 90%, at least about 95% or at least about 99%.

The TNF antagonist preferably binds to TNFR. The TNF antagonist preferably specifically binds to TNFR. The TNF antagonist preferably specifically binds to TNFR 1. The TNF antagonist preferably specifically binds to TNFR 2. The TNF antagonist preferably specifically binds to TNFR 1 and TNFR 2. The TNF antagonist preferably reduces the ability of TNFR to trimerise. The TNF antagonist preferably prevents TNFR from trimerising. The TNF antagonist preferably reduces the ability of TNFR to be activated by TNF. The TNF antagonist preferably prevents TNFR being activated by TNF. The TNF antagonist preferably reduces the ability of TNFR to carry out intracellular signalling. The TNF antagonist preferably prevents TNFR from carrying out intracellular signalling.

The TNF antagonist preferably reduces the bioavailability of TNFR. The TNF antagonist preferably reduces the bioavailability of TNFR by from about 1 to about 100%, such as at least about 50%, at least about 60%, at least about 70%, at least about 80%, at least about 90%, at least about 95% or at least about 99%. The TNF antagonist preferably sequesters the TNFR such that it is not available to bind to TNF.

The TNF antagonist preferably binds to the TNF-TNFR complex. The TNF antagonist preferably prevents the activation of the TNF-TNFR complex.

Decreasing the activity of TNF and/or TNFR may take place via any suitable mechanism, depending for example on the nature (see below) of the TNF antagonist used, e.g. steric interference in any direct or indirect TNF TNFR interaction or knockdown of TNF and/or TNFR expression.

The TNF antagonist preferably decreases the expression of TNF. Decreasing the expression of TNF encompasses decreasing the transcription of TNF mRNA. Decreasing the expression of TNF encompasses decreasing the trafficking of TNF mRNA out of the nucleus. Decreasing the expression of TNF encompasses decreasing the translation of TNF protein. Decreasing the expression of TNF encompasses decreasing the post-translational modification of TNF protein. Decreasing the expression of TNF encompasses decreasing the trafficking of TNF protein to the cell membrane. Decreasing the expression of TNF encompasses decreasing the expression of TNF protein at the cell membrane. Decreasing the expression of TNF encompasses decreasing the cleavage of TNF protein from the cell membrane. Decreasing the expression of TNF encompasses increasing the degradation of TNF.

The TNF antagonist preferably decreases the expression of TNF by from about 1 to about 100%, such as at least about 50%, at least about 60%, at least about 70%, at least about 80%, at least about 90%, at least about 95% or at least about 99%.

The TNF antagonist preferably decreases the expression of TNFR. Decreasing the expression of TNFR encompasses decreasing the transcription of TNFR mRNA. Decreasing the expression of TNFR encompasses decreasing the trafficking of TNFR mRNA out of the nucleus. Decreasing the expression of TNFR encompasses decreasing the translation of TNFR protein. Decreasing the expression of TNFR encompasses decreasing the post-translational modification of TNFR protein. Decreasing the expression of TNFR encompasses decreasing the trafficking of TNFR protein to the cell membrane. Decreasing the expression of TNFR encompasses decreasing the expression of TNFR protein at the cell membrane. Decreasing the expression of TNFR encompasses increasing the degradation of TNFR.

The TNF antagonist preferably decreases the expression of TNFR by about 1 to about 100%, such as at least about 50%, at least about 60%, at least about 70%, at least about 80%, at least about 90%, at least about 95% or at least about 99%.

Decreasing the expression of TNF and/or TNFR may take place via any suitable mechanism, depending for example on the nature (see below) of the TNF antagonist used.

The TNF antagonist preferably (a) decreases the activity of TNF, (b) decreases the activity of TNFR, (c) decreases the expression of TNF or (d) decreases the expression of TNFR. The TNF antagonist may perform any combination of (a) to (d), such as (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) or (d).

Decreasing the activity of TNF and/or TNFR and/or the expression of TNF and/or TNFR can be measured by any suitable means. For example, prevention of the TNF-TNFR interaction can be determined by a TNF-TNFR binding assay or an IK-β phosphorylation assay. Expression of TNF and/or TNFR may be measured using immunohistochemistry, western blotting, mass spectrometry, fluorescence-activated cell sorting (FACS) or quantitative reverse transcription polymerase chain reaction (qRT-PCR), such as real time qRT-PCR.

Any suitable antagonist may be used according to the invention. For example a small molecule, a polypeptide, a peptide or peptidomimetic, a polynucleotide, an oligonucleotide, an antisense RNA, small interfering RNA (siRNA) or small hairpin RNA (shRNA), an aptamer, or an antibody or antibody fragment may be used. Preferred antagonists include, but are not limited to, peptide fragments of TNF or TNFR, double-stranded RNA, aptamers and antibodies.

### Small Molecules

Small molecule TNF antagonists for use in the invention encompass any known in the art. Examples of small molecule TNF antagonists include N-alkyl 5-arylidene-2-thioxo-1,3-thiazolidin-4-ones, particularly IW927 and IV703. Other examples include LMP-420 and Japonicone A. Examples of small molecule modulators of TNF production include pentoxyfilline or bupropion.

### Peptides and Polypeptides

The TNF antagonist for use in the invention can comprise a peptide. The TNF antagonist for use in the invention can comprise a peptidomimetic. The TNF antagonist for use in the invention can comprise a polypeptide. A peptide is a polymer comprising from about two to about 50 amino acids. A polypeptide is a polymer comprising about 50 or more amino acids. The amino acids can be naturally occurring or artificial. One or more of the amino acids in the peptide or polypeptide may be D amino acids. Said peptides and polypeptides may be linear or cyclic.

Peptide and polypeptide TNF antagonists will typically comprise fragments of TNF. Such fragments may compete with full-length TNF for binding to TNFR and hence antagonise TNF. Peptide and polypeptide antagonists will typically comprise fragments of TNFR. Peptide and polypeptide antagonists will typically comprise fragments of TNFR that bind to TNF and hence antagonise TNF. Peptide and polypeptide TNF antagonists may bind to TNF and so prevent the binding of TNF to TNFR. Peptide and polypeptide TNF antagonists may bind to TNFR and so prevent the binding of TNF to TNFR. Peptide and polypeptide TNF antagonists may bind to and sequester TNF. Peptide and polypeptide TNF antagonists may bind to and sequester TNFR.

The TNF antagonist preferably comprises at least about 10 consecutive amino acids from SEQ ID NO: 3 or 4 or a variant thereof having at least about 80%, at least about 90%, at least about 95%, at least about 98% or at least about 99% amino acid sequence identity to SEQ ID NOs: 3 or 4 over its entire length. The TNF antagonist preferably comprise at least about 10 consecutive amino acids from SEQ ID NOs: 7 or 10 or a variant thereof having at least about 80%, at least about 90%, at least about 95%, at least about 98% or at least about 99% amino acid sequence identity to SEQ ID NOs: 7 or 10 over its entire length. The TNF antagonist may comprise at least about 20, at least about 30, at least about 40, at least about 50, at least about 60, at least about 70, at least about 80, at least about 90 or at least about 100 consecutive amino acids from SEQ ID NOs: 3, 4, 7 or 10 or a variant thereof having at least about 80%, at least about 90%, at least about 95%, at least at least about 98% or at least about 99% amino acid sequence identity to SEQ ID NOs: 3, 4, 7 or 10 over its entire length.

Peptide and polypeptide TNF antagonists can be identified in any suitable manner, for example, by systematic screening of contiguous or overlapping peptides spanning part or all of the TNF or TNFR sequences. Peptidomimetics may also be designed to mimic such peptide antagonists.

The ability of a peptide or polypeptide to bind TNF and/or TNFR can be determined using any method. Binding assays for TNF are known in the art. For instance, dissociation constants may be measured using radioactively labelled TNF and/or surface plasmon resonance techniques (see Grell et al., 1998, Eur. J. Immunol.)

The above mentioned sequence identity is calculated on the basis of amino acid identity. The UWGCG Package provides programs including GAP, BESTFIT, COMPARE, ALIGN and PILEUP that can be used to calculate sequence identity or to line up sequences (for example used on their default settings). The BLAST algorithm can also be used to compare or line up two sequences, typically on its default settings. Software for performing a BLAST comparison of two sequences is publicly available through the National Center for Biotechnology Information (http://www.ncbi.nlm.nih.gov/). This algorithm is further described below. Similar publicly available tools for the alignment and comparison of sequences may be found on the European Bioinformatics Institute website (http://www.ebi.ac.uk), for example the ALIGN and CLUSTALW programs.

A BLAST analysis is preferably used for calculating identity. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Similar sequences typically differ by at least 1, 2, 5, 10, 20 or more mutations (which may be substitutions, deletions or insertions of amino acids). These mutations may be measured across any of the regions mentioned above in relation to calculating identity. In the case of proteins as above, the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Any of the peptide and polypeptide TNF antagonists for use in the invention may be in the form of a dimer. Any of the peptide and polypeptide TNF antagonists may further be chemically-modified to form a derivative. Derivatives include peptides or polypeptides that have lipid extensions or have been glycosylated. Derivatives also include peptides or polypeptides that have been detectably labelled. Detectably labelled peptides or polypeptides have been labelled with a labelling moiety that can be readily detected. Examples of labelling moieties include, but are not limited to, radioisotopes or radionucleotides, fluorophores such as green fluorescent protein (GFP), electron-dense reagents, quenchers of fluorescence, enzymes, affinity tags and epitope tags. Preferred radioisotopes include tritium and iodine. Affinity tags are labels that confer the ability to specifically bind a reagent onto the labelled molecule. Examples include, but are not limited to, biotin, histidine tags and glutathione-S-transferase (GST). Labels may be detected by, for example, spectroscopic, photochemical, radiochemical, biochemical, immunochemical or chemical methods that are known in the art.

Any of the peptide and polypeptide TNF antagonists for use in the invention may also comprise additional amino acids or polypeptide sequences. The peptide and polypeptide TNF antagonists may comprise additional polypeptide sequences such that they form fusion proteins. The additional polypeptide sequences may be fused at the amino terminus, carboxy terminus or both the amino terminus and the carboxy terminus. Examples of fusion partners include, but are not limited to, GST, maltose binding protein, alkaline phosphatates, thiorexidin, GFP, histidine tags and epitope tags (for example, Myc or FLAG).

Peptide and polypeptide TNF antagonists for use in the invention may also comprise soluble TNF receptor chimeras. Examples of soluble TNF receptor chimeras include, but are not limited to, pegsunercept, lenercept and etanercept.

Etanercept, known as Enbrel®, binds to TNF and decreases its role in diseases involving excess inflammation in humans and other animals, including autoimmune diseases such as ankylosing spondylitis, juvenile rheumatoid arthritis, psoriasis, psoriatic arthritis, rheumatoid arthritis, and, potentially, in a variety of other diseases mediated by excess TNF.

Peptides and polypeptides may be made using standard methods. The polynucleotide sequence encoding a peptide or polypeptide may be cloned into any suitable expression vector. In an expression vector, the polynucleotide sequence encoding the peptide or polypeptide is typically operably linked to a control sequence which is capable of providing for the expression of the coding sequence by the host cell. Such expression vectors can be used to express the peptide or polypeptide.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, the peptide or polypeptide can be produced by inserting a polynucleotide sequence encoding a construct into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the peptide or polypeptide at a high level. Host cells transformed with a polynucleotide sequence encoding the peptide or polypeptide will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

### Polynucleotides and Oligonucleotides

The TNF antagonist for use in the invention can comprise a polynucleotide. The TNF antagonist for use in the invention can comprise an oligonucleotide. The TNF antagonist for use in the invention can comprise an antisense RNA. The TNF antagonist for use in the invention can comprise a small interfering RNA (siRNA). The TNF antagonist for use in the invention can comprise a small hairpin RNA (shRNA).

Polynucleotide and oligonucleotide TNF antagonists for use in the invention may decrease the expression of TNF or TNFR. Polynucleotide and oligonucleotide TNF antagonists for use in the invention may bind to or hybridise to TNF or TNFR mRNAs. Polynucleotide and oligonucleotide TNF antagonists for use in the invention may bind to or hybridise to TNF mRNA as shown in SEQ ID NO: 2 or TNFR mRNA, as shown in SEQ ID NO: 6 or 9, or any allelic variants thereof.

The terms “nucleic acid molecule” and “polynucleotide” are used interchangeably herein and refer to a polymeric form of nucleotides of any length, either deoxyribonucleotides, ribonucleotides or analogs thereof. The terms “nucleic acid molecule” and “oligonucleotide” are used interchangeably and refer to a polymeric form of nucleotides, either deoxyribonucleotides, ribonucleotides or analogs thereof, of fewer than about 100 or about 50 nucleotides. A nucleic acid may comprise conventional bases, sugar residues and inter-nucleotide linkages, but may also comprise modified bases, modified sugar residues or modified linkages. A polynucleotide or oligonucleotide may be single stranded or double stranded.

The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide or oligonucleotide can be oxidized or methylated. One or more nucleotides in the polynucleotide or oligonucleotide may be damaged. For instance, the polynucleotide or oligonucleotide may comprise a pyrimidine dimer. Such dimers are typically associated with damage by ultraviolet light.

The nucleotides in the polynucleotide or oligonucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide or oligonucleotide can be a nucleic acid, such as deoxyribonucleic acid (DNA) or a ribonucleic acid (RNA). The polynucleotide or oligonucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide or oligonucleotide may be single stranded or double stranded.

The TNF antagonist may comprise an oligonucleotide or polynucleotide which specifically hybridises to a part of SEQ ID NO: 2 (TNF mRNA), or any allelic variant thereof. The oligonucleotide is preferably RNA.

The TNF antagonist may comprise an oligonucleotide or polynucleotide which specifically hybridises to a part of SEQ ID NO: 6 (TNFR 1 mRNA), or any allelic variant thereof.

The TNF antagonist may comprise an oligonucleotide or polynucleotide which specifically hybridises to a part of SEQ ID NO: 9 (TNFR 2 mRNA), or any allelic variant thereof.

The oligonucleotide is preferably RNA.

A “part” can be defined as any number of consecutive nucleotides from TNF or TNFR mRNA, such as SEQ ID NO: 2, 6 or 9. A part may be about 10, 15, 20, 25, 30, 40, 50, 100, 150, 200 or more consecutive nucleotides. A part may be about 20, 21, 22, 23, 24 or 25 consecutive nucleotides of TNF or TNFR mRNA. A part may be about 10, 11, 12, 13, 14 or 15 consecutive nucleotides of TNF or TNFR mRNA.

The length of the oligonucleotide or polynucleotide is not fixed, as long as the oligonucleotide or polynucleotide is capable of binding to TNF or TNFR mRNA. The oligonucleotide may comprise a nucleic acid sequence that is complementary to a part of the TNF or TNFR mRNA sequence and may further comprise additional nucleotides at the 5′ and/or 3′ ends of that complementary sequence.

The polynucleotide TNF antagonist may be about 500, 400, 300, 200 or 100 nucleotides/nucleotide pairs or fewer in length.

The oligonucleotide or polynucleotide TNF antagonist may be about 100, 90, 80, 70, 60, 50, 40, 30, 25 or 20 nucleotides/nucleotide pairs or fewer in length. The oligonucleotide or polynucleotide may be at least about 20, 21, 22, 23, 24, 25, 26 or 27 nucleotides/nucleotide pairs in length, preferably at least about 23, 24, 25 or 26 nucleotides/nucleotide pairs in length, especially about 25 nucleotides/nucleotide pairs in length. The oligonucleotide or polynucleotide TNF antagonist is preferably about 20, 21 or 22 nucleotides/nucleotide pairs in length.

The oligonucleotide or polynucleotide TNF antagonist may be capable of specifically binding, or specifically hybridising, to the mRNA sequence of TNF or TNFR as set forth in SEQ ID NOs: 2, 6 and 9, or allelic variants, but not other mRNA sequences.

An oligonucleotide or polynucleotide “specifically hybridises” to a target sequence when it hybridises with preferential or high affinity to the target sequence but does not substantially hybridise, does not hybridise or hybridises with only low affinity to other sequences.

An oligonucleotide or polynucleotide “specifically hybridises” if it hybridises to the target sequence with a melting temperature (Tₘ) that is at least 2° C., such as at least 3° C., at least 4° C., at least 5° C., at least 6° C., at least 7° C., at least 8° C., at least 9° C. or at least 10° C., greater than its Tₘ for other sequences. More preferably, the oligonucleotide or polynucleotide hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for other nucleic acids. Preferably, the portion hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for a sequence which differs from the target sequence by one or more nucleotides, such as by 1, 2, 3, 4 or 5 or more nucleotides. The portion typically hybridises to the target sequence with a T of at least 90° C., such as at least 92° C. or at least 95° C. Tₘ can be measured experimentally using known techniques, including the use of DNA microarrays, or can be calculated using publicly available Tₘ calculators, such as those available over the internet.

Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-lnterscience, New York (1995)). Hybridisation can be carried out under low stringency conditions, for example in the presence of a buffered solution of 30 to 35% formamide, 1 M NaCl and 1 SDS (sodium dodecyl sulfate) at 37° C. followed by a 20 wash in from 1× (0.1650 M Na⁺) to 2× (0.33 M Na⁺) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na⁺) to 1× (0.1650 M Na⁺) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na⁺) SSC at 60° C.

The oligonucleotide or polynucleotide may comprise a sequence which is substantially complementary to the target sequence. Typically, the oligonucleotides or polynucleotides are 100% complementary. However, lower levels of complementarity may also be acceptable, such as 95%, 90%, 85% and even 80%. Complementarity below 100% is acceptable as long as the oligonucleotides specifically hybridise to the target sequence. An oligonucleotide or polynucleotide may therefore have 1, 2, 3, 4, 5, 6, 7, 8, 9, 10 or more mismatches across a region of 5, 10, 15, 20, 21, 22, 30, 40 or 50 nucleotides.

Using known techniques and based on a knowledge of the sequence of TNF or TNFR, double-stranded RNA (dsRNA) molecules can be designed to antagonise TNF by sequence identity-based targeting of TNF RNA, as set forth in SEQ ID NO: 2, or TNFR RNA, as set forth in SEQ ID NOs: 6 or 9. Such dsRNAs will typically be small interfering RNAs (siRNAs), usually in a stem-loop (“hairpin”) configuration, or micro-RNAs (miRNAs). The sequence of such dsRNAs will comprise a portion that corresponds with that of a portion of the mRNA encoding TNF or TNFR. This portion will usually be 100% complementary to the target portion within the TNF or TNFR mRNA but lower levels of complementarity (e.g. at least about 85%, 90% or more or 95% or more) may also be used.

The TNF antagonist may comprise an oligonucleotide which comprises about 50 or fewer consecutive nucleotides, such as about 40 or fewer, about 30 or fewer or about 25 or fewer consecutive nucleotides, from SEQ ID NO: 1 or a variant thereof which has at least about 90% identity, such as at least about 95% identity, at least about 96% identity, at least about 97% identity, at least about 98% identity or at least about 99% identity, to SEQ ID NO: 1 over its entire sequence. The oligonucleotide is preferably about 20, 21, 22, 23, 24 or 25 nucleotides in length. The oligonucleotide is preferably RNA. The oligonucleotide may be double stranded and may be any of the aforementioned lengths where nucleotides are replaced with nucleotide pairs.

The TNF antagonist may comprise an oligonucleotide which comprises about 50 or fewer consecutive nucleotides, such as about 40 or fewer, about 30 or fewer or about 25 or fewer consecutive nucleotides, from SEQ ID NOs: 5 or 8 or a variant thereof which has at least about 90% identity, such as at least about 95% identity, at least about 96% identity, at least about 97% identity, at least about 98% identity or at least about 99% identity, to SEQ ID NOs: 5 or 8 over its entire sequence. The oligonucleotide is preferably about 20, 21, 22, 23, 24 or 25 nucleotides in length. The oligonucleotide may be double stranded and may be any of the aforementioned lengths where nucleotides are replaced with nucleotide pairs.

Oligonucleotide or polynucleotide sequences may be isolated and replicated using standard methods in the art. The gene encoding a TNF antagonist of the invention may be amplified using PCR involving specific primers. The amplified sequences may then be incorporated into a recombinant replicable vector such as a cloning vector. The vector may be used to replicate the polynucleotide in a compatible host cell. Thus polynucleotide or oligonucleotide sequences encoding the TNF antagonist for use in the invention may be made by introducing a polynucleotide or oligonucleotide encoding the TNF antagonist for use in the invention into a replicable vector, introducing the vector into a compatible host cell, and growing the host cell under conditions which bring about replication of the vector. The vector may be recovered from the host cell. Suitable host cells for cloning of polynucleotides and oligonucleotides are known in the art and described in more detail above.

### Aptamers

Aptamers are generally nucleic acid molecules that bind a specific target molecule. The aptamers for use in the present invention preferably bind to specific target molecules and therefore block the function of TNF. The aptamers for use in the present invention preferably bind to TNF or TNFR.

Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Non-modified aptamers are cleared rapidly from the bloodstream, with a half-life of minutes to hours, mainly due to nuclease degradation and clearance from the body by the kidneys. Such non-modified aptamers have utility in, for example, the treatment of transient conditions such as in stimulating blood clotting. Alternatively, aptamers may be modified to improve their half life. Several such modifications are available, such as the addition of 2′-fluorine-substituted pyrimidines or polyethylene glycol (PEG) linkages.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. No. 5,654,151, U.S. Pat. No. 5,503,978, U.S. Pat. No. 5,567,588 and WO 96/38579.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Antibodies

The TNF antagonist preferably comprises an antibody or antibody fragment. Antibody TNF antagonists may bind to TNF and so prevent the binding of TNF to TNFR. Antibody TNF antagonists may bind to TNFR and so prevent the binding of TNF to TNFR. Antibody TNF antagonists may bind to and sequester TNF. Antibody TNF antagonists may bind to and sequester TNFR. The TNF antagonist preferably comprises an antibody or antibody fragment which specifically binds to TNF, TNFR or any allelic variant thereof. The TNF antagonist preferably comprises an antibody or antibody fragment which specifically binds to TNF and TNFR or any allelic variants thereof. TNF preferably comprises the sequence shown in SEQ ID NOs: 3 or 4. TNFR preferably comprises the sequence shown in SEQ ID NOs: 7 or 10.

An antibody, or other compound, “specifically binds” to a polypeptide when it binds with preferential or high affinity to the protein for which it is specific, but does substantially bind, not bind or binds with only low affinity to other polypeptides. The TNF antagonist may be capable of specifically binding the amino acid sequence of TNF or TNFR as set forth in SEQ ID NOs: 3, 4, 7 and 10, or allelic variants, but not other proteins.

For example, an antibody specifically binds to an antigen if it binds to the antigen with preferential or high affinity, but does not bind or binds with only low affinity to other or different antigens. An antibody binds with preferential or high affinity if it binds with a Kd of 1×10⁻⁶ M or less, more preferably 1×10⁻⁷ M or less, 5×10⁻⁸ M or less, more preferably 1×10⁻⁸ M or less or more preferably 5×10⁻⁹ M or less. An antibody binds with low affinity if it binds with a Kd of 1×10⁻⁶ M or more, more preferably 1×10⁻⁵ M or more, more preferably 1×10⁻⁴ M or more, more preferably 1×10⁻³ M or more, even more preferably 1×10⁻² M or more.

A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. Each light chain is comprised of a light chain variable region (abbreviated herein as V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR).

The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies.

Antibodies for use in the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising an antibody in a host animal against the whole polypeptide or a fragment thereof, for example an antigenic epitope thereof, hereinafter the “immunogen”. The fragment may be any of the fragments mentioned herein (typically at least 10 or at least 15 amino acids long).

An antibody for use in the invention may be a monoclonal antibody or a polyclonal antibody, and will preferably be a monoclonal antibody. An antibody of the invention may be a chimeric antibody, a CDR-grafted antibody, a nanobody, a human or humanised antibody or an antigen binding portion of any thereof. For the production of both monoclonal and polyclonal antibodies, the experimental animal is typically a non-human mammal such as a goat, rabbit, rat or mouse but may also be raised in other species such as camelids.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

Monoclonal antibodies (mAbs) for use in the invention can be produced by a variety of techniques, including conventional monoclonal antibody methodology e.g., the standard somatic cell hybridization technique of Kohler and Milstein. The preferred animal system for preparing hybridomas is the murine system. Hybridoma production in the mouse is a very well-established procedure and can be achieved using techniques well known in the art.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An antibody for use in the invention may be produced by a method comprising: immunising a non-human mammal with an immunogen comprising full-length TNF or a peptide fragment of TNF, obtaining an antibody preparation from said mammal; and deriving therefrom monoclonal antibodies that specifically recognise said epitope.

An antibody for use in the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody for use in the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies for use in the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

Antibodies for use in the invention will have TNF antagonist properties as discussed above. In one embodiment, a monoclonal antibody specifically recognises an epitope within TNF and blocks the activity of TNF. In anther embodiment, a monoclonal antibody specifically recognises an epitope within TNFR and blocks the activity of TNFR. In one embodiment, the monoclonal antibody specifically recognises an epitope within TNF and blocks the interaction between TNF and TNFR. In one embodiment, the monoclonal antibody specifically recognises an epitope within TNFR and blocks the interaction between TNF and TNFR.

Antibodies for use in the invention can be tested for binding to TNF or TNFR by, for example, standard ELISA or Western blotting. An ELISA assay can also be used to screen for hybridomas that show positive reactivity with the target protein. The binding specificity of an antibody may also be determined by monitoring binding of the antibody to cells expressing the target protein, for example by flow cytometry.

Examples of antibodies for use in the invention are infliximab, adalimumab, certolizumab, certolizumab pegol, and golimumab. Infliximab and adalimumab are examples of antibodies capable of neutralising all forms (extracellular, transmembrane, and receptor-bound) of TNF. Accordingly, in a preferred aspect of the invention, the TNF antagonists comprise infliximab and/or adalimumab.

Infliximab (sold under the brand name Remicade®) is a drug used to treat inflammatory and autoimmune diseases. Infliximab is a chimeric monoclonal antibody comprising murine binding VK and VH domains and human constant Fc domains.

Infliximab neutralizes the biological activity of TNF by binding with high affinity to the soluble (free floating in the blood) and transmembrane (located on the outer membranes of T cells and similar immune cells) forms of TNF and inhibits or prevents the effective binding of TNF with its receptors. Infliximab has high specificity for TNF, and does not neutralize LT-alpha, although LT-alpha utilizes the same receptors as TNF. Infliximab has been approved by the U.S. Food and Drug Administration for the treatment of, for example, psoriasis, paediatric Crohn's disease, ankylosing spondylitis, Crohn's disease, psoriatic arthritis, rheumatoid arthritis, and ulcerative colitis.

Adalimumab (sold under the brand name Humira®) also binds to TNF, preventing it from activating TNF receptors. Adalimumab was constructed from a fully human monoclonal antibody, while infliximab is a mouse-human chimeric antibody. Adalimumab has been approved by the United States Food and Drug Administration (FDA) for the treatment of, for example, rheumatoid arthritis, psoriatic arthritis, alkylosing sponylitis and Crohn's disease, and an application has been made to the FDA for approval in the treatment of plaque psoriasis.

In another preferred aspect of the invention, the TNF antagonist comprises a neutralising antibody to a TNF receptor. Typically, the neutralising antibody to the TNF receptor is a neutralising antibody to TNFR as set forth in SEQ ID NOs: 7 or 10. Examples of neutralising antibodies to the TNFR receptor include, but are not limited to, atrosab. Atrosab binds to amino acids 1 to 70 of human TNFR1 and selectively inhibits TNFR1-mediated signal transduction (see Zettlitz et al. (2010) MAbs 2:6, 639-647).

Method of treating a LSD

The present invention relates to a method of treating a LSD in a patient, wherein the method comprises administering to the patient a TNF antagonist and thereby treating the LSD.

Methods of administration and conditions to be treated are described below. The dose of the TNF antagonist to be used in accordance with the invention will depend upon the nature of the LSD. A suitable dose can be determined by a skilled practitioner based on his common general knowledge, taking into account, for example, the mode of administration. For example, a suitable dose may be selected to reflect the level of a therapeutic agent that would be present in the blood circulatory system of a patient after in vivo administration.

The TNF antagonist can be administered to the patient by any suitable means. The TNF antagonist can be administered by enteral or parenteral routes such as via oral, buccal, anal, pulmonary, intravenous, intra-arterial, intramuscular, intraperitoneal, intraarticular, topical or other appropriate administration routes.

The formulation will depend upon the TNF antagonist. The TNF antagonist may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally or by infusion techniques. The TNF antagonist may also be administered as a suppository. A physician will be able to determine the required route of administration for each particular patient.

The TNF antagonist can be formulated for use with a pharmaceutically acceptable carrier or diluent and this may be carried out using routine methods in the pharmaceutical art. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Pharmaceutical compositions suitable for delivery by needleless injection, for example, transdermally, may also be used.

The method of the invention may be for treating a LSD. In the case of treating, the patient typically has the LSD, i.e. has been diagnosed as having the LSD, or is suspected as having the LSD, i.e. shows the symptoms of the LSD. As used herein, the term “treating” includes any of following: the prevention of the LSD or of one or more symptoms associated with the LSD; a reduction or prevention of the development or progression of the LSD or symptoms; and the reduction or elimination of an existing LSD or symptoms. Symptoms as described herein include neurological symptoms. Examples of neurological symptoms include ataxia, dystonia, vertical supranuclear gaze palsy and dementia.

The method may be for preventing the disease. In this embodiment, the patient can be asymptomatic. The patient can have a genetic predisposition to the LSD. The patient may have one or more family member(s) with an LSD. As used herein, the term “preventing” includes the prevention of the onset of the disease or of one or more symptoms associated with the disease. Symptoms as described herein include neurological symptoms. Examples of neurological symptoms include ataxia, dystonia, vertical supranuclear gaze palsy and dementia.

Therapy and prevention includes, but is not limited to, preventing or eliciting an effective anti-TNF response and/or preventing, alleviating, reducing, curing or at least partially arresting symptoms and/or complications resulting from or associated with an LSD. When provided therapeutically, the therapy is typically provided at or shortly after the onset of a symptom of the LSD. Such therapeutic administration is typically to prevent or ameliorate the progression of, or a symptom of the LSD or to reduce the severity of such a symptom or LSD. When provided prophylactically, the treatment is typically provided before the onset of a symptom of an LSD. Such prophylatic administration is typically to prevent the onset of symptoms of the LSD. Symptoms as described herein include neurological symptoms. Examples of neurological symptoms include ataxia, dystonia, vertical supranuclear gaze palsy and dementia.

Specific routes, dosages and methods of administration of TNF antagonists for use in the invention may be routinely determined by the medical practitioner. Typically, a therapeutically effective or a prophylactically effective amount of the TNF antagonist is administered to the patient. A prophylactically effective amount is an amount which prevents the onset of one or more symptoms of the LSD. A therapeutically effective amount of the compound is an amount effective to ameliorate one or more symptoms of the LSD.

A therapeutically or prophylactically effective amount of the TNF antagonist is administered. The dose may be determined according to various parameters, especially according to the compound used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the patient to be treated, the type and severity of the disease and the frequency and route of administration. Preferably, daily dosage levels are from 5 mg to 2 g.

For example, infliximab is currently sold under the brand name Remicade®. Remicade® is sold for administration by intravenous infusion, typically at 2-month intervals and at a clinic or hospital. The recommended dose of Remicade® for treatment of rheumatoid arthritis is 3 mg/kg, followed by additional similar doses at 2 and 6 weeks after the first infusion and then every 8 weeks thereafter. For Crohn's disease, alkylosing spondylitis, psoriatic arthritis, psoriasis or ulcerative colitis, the recommended dose of Remicade® is 5 mg/kg over a similar administration schedule.

Adalimumab is currently sold under the brand name Humira®. Humira® is marketed in both preloaded 0.8 ml syringes and in preloaded pen devices, both for injection subcutaneously, typically by the patient at home. In accordance with the present invention, TNF antagonists may be administered via any suitable route in any suitable dose and in any suitable administration regime. The recommended dose of Humira® for adult patients with rheumatoid arthritis, psoriatic arthritis or ankylosing spondylitis is 40 mg administered every other week. The recommended Humira® dose regimen for adult patients with Crohn's disease is 160 mg initially at Week 0 (dose can be administered as four injections in one day or as two injections per day for two consecutive days), 80 mg at Week 2, followed by a maintenance dose of 40 mg every other week beginning at Week 4.

The TNF antagonist may be a polynucleotide. Preferably, such polynucleotides are provided in the form of an expression vector, which may be expressed in the cells of the patient to be treated. The polynucleotides may be naked nucleotide sequences or be in combination with cationic lipids, polymers or targeting systems. The polynucleotides may be delivered by any available technique. For example, the polynucleotide may be introduced by needle injection, preferably intradermally, subcutaneously or intramuscularly. Alternatively, the polynucleotide may be delivered directly across the skin using a polynucleotide delivery device such as particle-mediated gene delivery. The polynucleotide may be administered topically to the skin, or to mucosal surfaces for example by intranasal, oral, intravaginal or intrarectal administration.

Uptake of polynucleotide constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the polynucleotide is administered in the range of 1 pg to 1 mg, preferably to 1 pg to 10 μg nucleic acid for particle mediated gene delivery and 10 μg to 1 mg for other routes.

The TNF antagonist may be employed alone as part of a composition, such as but not limited to a pharmaceutical composition or a vaccine composition or an immunotherapeutic composition to prevent and/or treat a LSD.

### Combination Therapies

A TNF antagonist may be used in combination with one or more other therapies intended prevent and/or to treat the LSD in the same patient. By a “combination” is meant that the therapies may be administered simultaneously, in a combined or separate form, to a patient. The therapies may be administered separately or sequentially to a patient as part of the same therapeutic regimen. The other therapy may be a general therapy aimed at treating or improving the condition of a patient with a LSD. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with a TNF antagonist. For example, infliximab is commonly used in combination with methotrexate in the treatment of rheumatoid arthritis.

The other therapy may be a specific treatment directed at the particular disease or condition suffered by the patient, or directed at a particular symptom of such a disease or condition. For example, where the patient has rheumatoid arthritis, the treatment may comprise treatment with a TNF antagonist, and also treatment with a further therapy specifically intended to treat, prevent or reduce the symptoms of the LSD.

### Kit

Also provided is a kit that comprises a means for diagnosing or prognosing a LSD and a TNF antagonist. In particular, such means may include a specific binding agent, probe, primer, pair or combination of primers, an enzyme or antibody, including an antibody fragment, as defined herein, which is capable of detecting or aiding in the detection of a LSD, as defined herein. The kit may comprise LysoTracker®, which is a fluorescent marker and is commercially-available from both Invitrogen and also Lonza. The LysoTracker® may be blue, blue-white, yellow, green or red. The chemical structures of LysoTracker® blue, green and red are shown in FIG. 8.

The kit also comprises a TNF antagonist. The kit may contain any of the TNF antagonists defined above. The kit may further comprise buffers or aqueous solutions. The kit may further comprise instructions for using the TNF antagonist in a method of the invention.

The invention is illustrated by the following Example:

### EXAMPLE

### Materials and Methods

Animals

BALB/c mice with a mutation in the NIH allele of the Niemann Pick type Cl gene (NPCnih) were bred as heterozygotes to produce Npc1−/− (BALB/c npcnih/nih) and control genotypes (Pentchev et al., 1980). Breeding and treatment of mice were conducted as described previously (Smith et al., 2009). The humane end point of mice was defined as a loss of 10% weight within a 24 hour time frame. All procedures were conducted according to Animals (Scientific Procedures) Act 1986 under a valid Home Office License.

### Drug Treatments

Enbrel® (Amgen, USA) was administered via subcutaneous injection (200 μg/animal; Koca et al., 2008) in phosphate-buffered saline (PBS) twice a week starting at 6 weeks of age (n=7). Control group animals (n=7) were administered equal volumes of PBS subcutaneously. Humira® was administered 20 mg/kg three times a week (doses converted from human doses based on differential binding coefficients against murine and human TNF) (n=4) and Remicade® 6.25 mg/kg once a week (Deveci et al., 2008) (n=5).

Animal Behavioural Studies

Body weights of individual mice were recorded on a weekly basis. Tremor and locomotor analysis of both treated and control groups were conducted as described by Smith et al. (2009). The procedures are briefly detailed as follows:

Locomotor Activity: AmLogger and Open Field Rearing

Spontaneous activity of each mouse was recorded weekly using both an automated activity monitor (AmLogger; Linton Instrumentation, UK) and manual counting (NPC mice have a jumping phenotype in late stages of disease that can be scored as rears in the automated system so manual counts were included for this reason). The activity monitor comprises an array of infrared beams to determine the activity and motility of the subject animal. After 5-minute acclimatization in the room, each mouse was placed in the ‘open-field’ (a plasticbox measuring 45×25×12 cm) with home cage bedding to be recorded for 5 minutes. A similar setup was used for the open field rearing study, where the number of times the mouse reared on its hind legs (either against the cage wall “rearing” or without support “centre rearing”) was counted manually for 5 minutes.

Tremor Analysis

A tremor monitor (used as per manufacturer's instructions; San Diego Instruments, USA) was placed on an anti-vibration table and the mice were monitored weekly. Animals were allowed to acclimatise for 30 seconds followed by a 256-second recording period. The output (amplitude/time) was analysed by fast Fourier transformation using LabView software (National Instruments, USA) and specified at each frequency from 31-51 Hz.

Gait Analysis

The CatWalk™ system (Noldus Information Technology, the Netherlands) was used for monitoring changes in movement patterns as indicative of any motor complications and to assess any gait improvement in response to treatment. Animals were subjected to CatWalk™ analysis biweekly, where a minimum of three ‘runs’ were collected per animal. The data collected were subsequently examined by CatWalk™ XT 10.0 software to produce a total of 177 inter-paw and intra-paw comparison parameters.

Data Analysis

All quantitative statistical analyses were performed using GraphPad Prism (GraphPad Software, USA) for validation. Comparisons were considered statistically significant when p<0.050 (*) using an unpaired, two-tailed Student's t-test. Inter-group comparisons were conducted using one-way analysis of variance (ANOVA) and Tukey test for pairwise multiple comparisons. All graphs have been constructed with mean values (minimum of three data points in each group) with standard errors shown unless where specified.

For basic survival parameters, rearing and tremor analysis, data from previous studies on related anti-TNF biologics, Humira® (Adalimumab) and Remicade® (Infliximab) (Kay et al., 2009; Shealy et al., 2010), have also been included for comparison.

### Example 1—Effect of TNF-Targeting Biologics on Survival of Npc1^{-/-} Mice

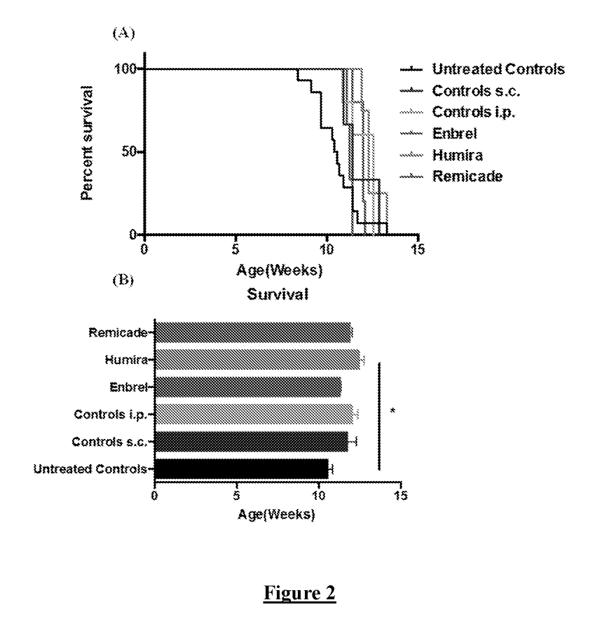

Upon treatment with 200 μm Enbrel® for twice a week starting at 7 weeks of age, Npc1^{-/-} mice were weighed weekly until humane end point to monitor any possible effects exerted by drug treatment. Enbrel® did not cause any significant difference in lifespan (11.26±0.09 weeks vs. 11.72±0.59 weeks; p=0.4840) of Npc1−/− mice (FIGS. 2A and B). Neither did Humira® or Remicade® impact mice survival (FIGS. 2A and B).

### Example 2—Effect of TNF-Targeting Biologics on Body Weight of Npc1^{-/-} Mice

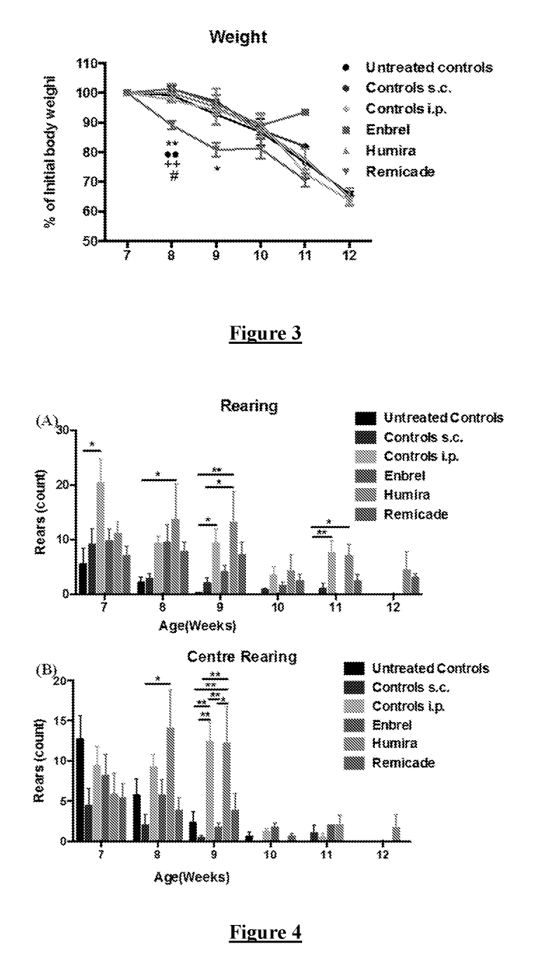

Upon treatment with 200 μg Enbrel® for twice a week starting at 7 weeks of age, Npc1^{-/-} mice were weighed weekly until humane end point to monitor any possible effects exerted by drug treatment. Enbrel® did not cause any significant difference in body weights (89.03% vs. 87.46% relative to respective initial weights at 7 weeks; p=0.7270) of Npc1^{-/-} mice (FIG. 3). Humira® did not impact mice weight. However, Remicade® caused significant weight loss at weeks 8 and 9 relative to initial weights at week 7 (89.11% vs. 101.31%; p<0.010 and 80.84% vs. 97.16%; p<0.010 respectively, relative to initial weight at week 7).

### Example 3—Effect of TNF-Targeting Biologics on (A) Rearing and (B) Centre Rearing of Npc1^{-/-} Mice

Neurodegeneration of NPC leads to cerebellar ataxia and various gait abnormalities. A range of behavioural parameters were monitored to assess the impact of the TNF blocker, Enbrel®, on NPC pathology and motor function.

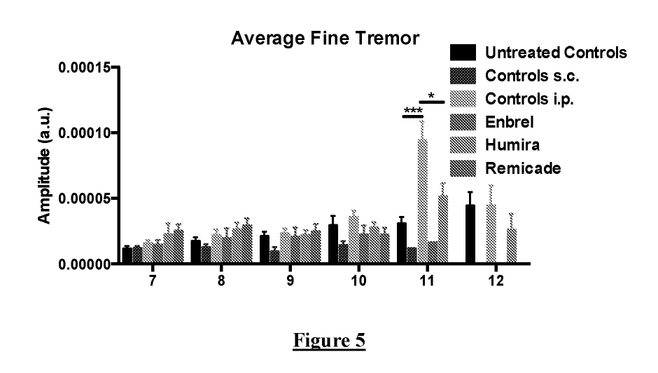

Activity levels of the mice (Npc1^{-/-}) were recorded and classified by either “rearing” (FIG. 4A; rearing sustained by front limb support on side walls of the open-field box) or “centre rearing” (FIG. 4B; rearing with no front-limb support). Overall, Enbrel®-treated mice displayed a higher level of rearing activity than control mice. In particular, rearing activity was maintained in Enbrel®-treated mice until 10 weeks whilst mice in the control group were no longer able to rear without front limb support (1.71 rears vs. 0.00 rears; p=0.01).

Similar beneficial effects were seen with Humira® on centre rearing at 8 and 9 weeks (10.00±3.58 rears vs. 2.00±1.35 rears; p=0.033 and 8.25±2.84 rears vs. 0.429±0.30 rears; p=0.005 respectively) and Remicade® on rearing at 8 and 9 weeks (7.80±1.71 rears vs. 2.86±1.03 rears; p=0.034 and 7.20±2.27 rears vs. 2.00±2.52 rears; p=0.039 respectively).

The Remicade® group presented with a sustained level of rearing activity starting from 7 weeks, higher than the two control groups (untreated and s.c. injected) and maintained up to 12 weeks.

Humira® was more effective amongst the other treatments starting from 8 weeks (p=0.037 vs untreated controls), at 9 weeks (p=0.002 and 0.021 vs untreated controls and the s.c. PBS injected controls) and at 11 weeks (p=0.011 vs untreated control). One way ANOVA with the Tukey test indicated that Humira® was significantly more effective than Enbrel® in retaining centre rearing function at 9 weeks (8.25±2.84 rears vs. 1.71±0.61 rears; p=0.025) while the reverse relationship can be seen at 10 weeks (1.71±0.57 rears vs. 0.00 rears; p=0.012).

Humira® treated mice sustained rearing activity beyond all control groups at later time points (FIG. 4B).

### Example 4—Effect of TNF-Targeting Biologics on Fine Tremor of Npc1^{-/-} Mice

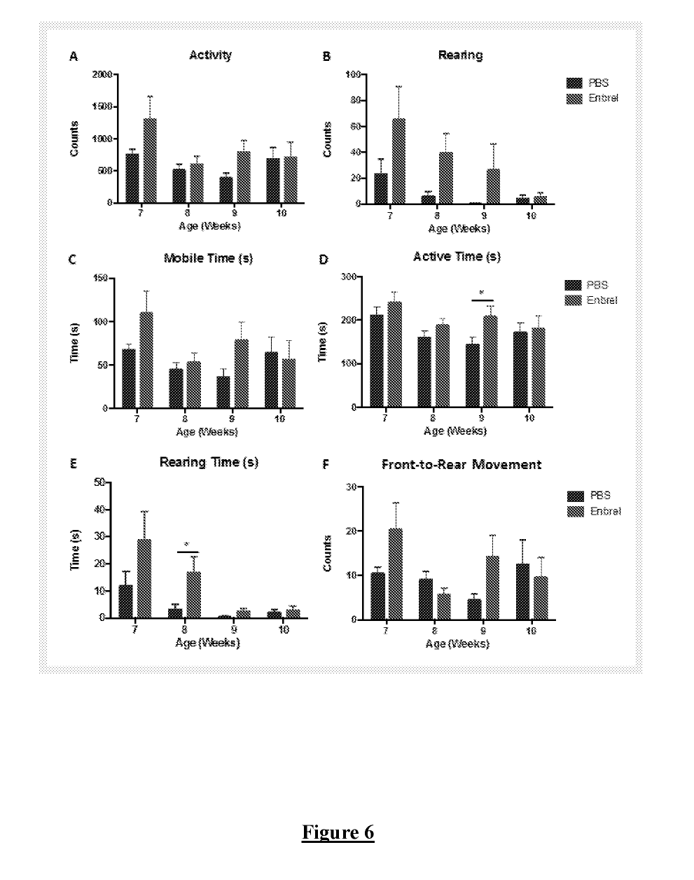

Progressive tremor is a feature of Npc1^{-/-} mice. Tremor amplitudes over the range of 31-51 Hz were recorded using a commercial tremor monitor. The average tremor intensity between 31-51 Hz was used to compare levels of fine tremor in experimental animals. All anti-TNF biologics had similar effects and did not have significant inter-treatment difference until 11 weeks, as validated by one-way ANOVA. The significantly higher tremor amplitude in the i.p. control group compared to untreated controls and the Humira group is indicative of a potential positive effect of Humira on tremor at this time point compared to i.p. saline only (FIG. 5).

### Example 5—Effect of Drug Treatment on Locomotor Activity

Automated activity measurements from the AmLogger system indicated that Enbrel® treatment induced higher levels of activity parameters, such as significantly longer times spent in an active state at 9 weeks (208.14±23.85 s vs. 143.43±17.625; p=0.0497) and in rearing at 8 weeks (16.86±5.79 s vs. 3.29±1.775; p=0.0447). Other measurements also demonstrated greater activity in mice following Enbrel® treatment relative to vehicle treated control Npc1^{-/-} mice (FIG. 6).

### Example 6—Effect of Drug Treatment on Single Paw Gait Parameters

Cerebellar ataxia following Purkinje cell loss results in gait abnormalities in NPC patients and animal models. The Noldus Catwalk™ system enables sensitive and quantitative analysis of paw patterns to reveal subtle changes in gait.

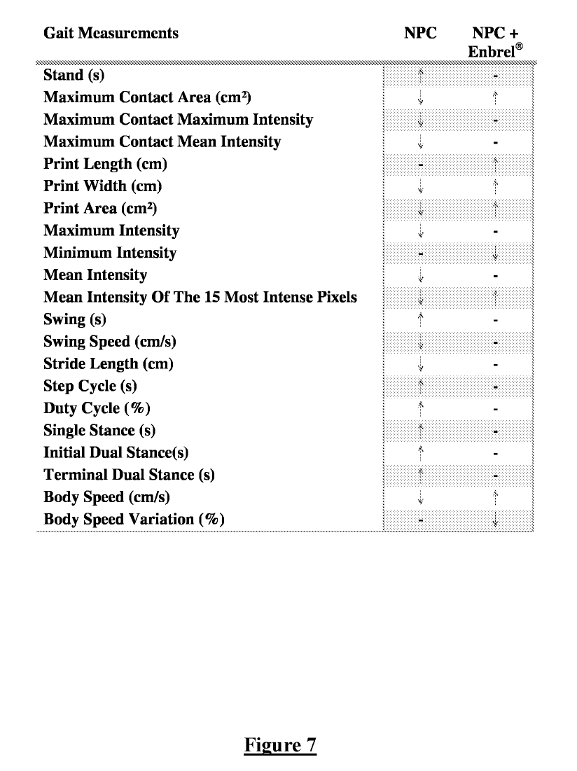

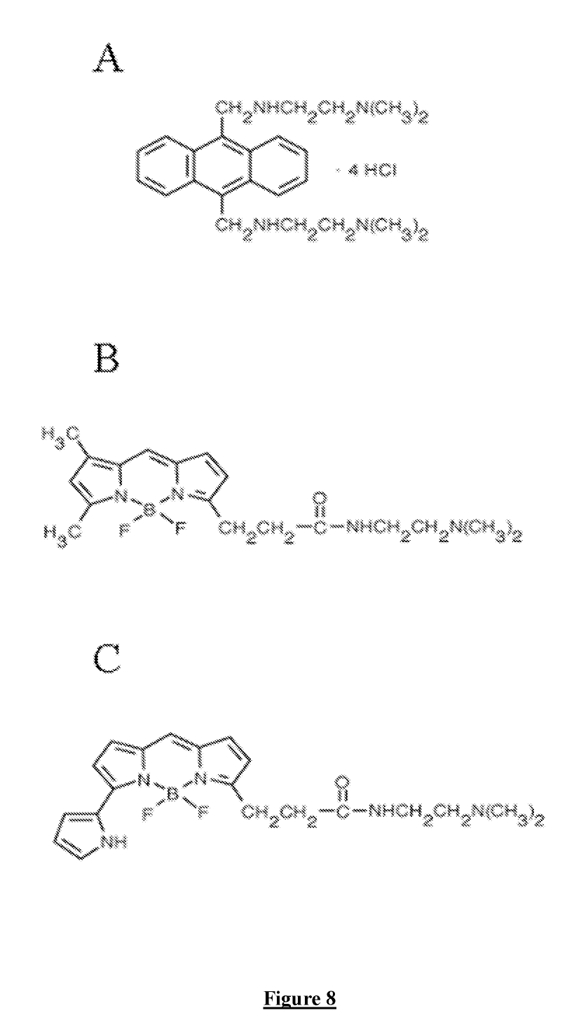

Previous studies have demonstrated that changes in single paw parameters occur earlier in NPC disease progression, showing significant difference by 7 weeks; whereas inter-paw differences only become significant by later stages of the diseases. For this reason, it can be assumed that single paw parameters would reveal more sensitive indications of treatment benefits in the short time frame allowed by this acute disease model. A summary of changes in intra-paw parameters following drug treatment has been tabulated in FIG. 7, indicating those parameters that were seen to be altered in NPC disease progression compared to wild type animals, as well as those that displayed trends upon Enbrel® treatment.

### REFERENCES

Patterson et al., (2006) Rev. Neurol. 43, 8
Grell et al., (1998), Eur. J. Immunol. 28, 257-263
Altschul et al., (1990) J Mol Biol. 215, 403-410
Henikoff and Henikoff (1992) Proc. Natl. Acad. Sci. USA 89, 10915-10919
Karlin and Altschul (1993) Proc. Natl. Acad. Sci. USA 90, 5873-5787
Maddox et al, (1993) J. Exp. Med. 158, 1211-1226
Kohler and Milstein (1975) Nature 256, 495-497
Zettlitz et al. (2010) MAbs 2, 639-647
Pentchev et al., (1980) J Biol Chem 261, 2772-2777
Smith et al., (2009) Neurobiol Dis. 36, 242-251
Koca et al., (2008) Rheumatology (Oxford) 47, 172-175.
Deveci et al., (2008) Respirology 13, 488-497
Kay et al., (2009) Core Evid. 4, 159-170
Shealy et al., (2010) MAbs 2 (4)

1. A method for preventing or treating a lysosomal storage disorder (LSD) in a patient, wherein the method comprises administering to the patient a Tumour Necrosis Factor (TNF) antagonist and thereby treating the LSD.

2. The method according to claim 1, wherein the TNF antagonist comprises a small molecule, a polypeptide, a peptide or peptidomimetic, a polynucleotide, an oligonucleotide, an antisense RNA, small interfering RNA (siRNA) or small hairpin RNA (shRNA), an aptamer, or an antibody or antibody fragment.

3. The method according to claim 1, wherein the TNF antagonist reduces the activity of TNF and/or Tumour Necrosis Factor Receptor (TNFR), or any allelic variant thereof.

4. The method according to claim 1, wherein the TNF antagonist comprises an antibody or antibody fragment which specifically binds to TNF, TNFR or any allelic variant thereof.

5. The method according to claim 4, wherein TNF comprises the sequence as shown in SEQ ID NOs: 3 or 4 or wherein TNFR comprises the sequence as shown in SEQ ID NOs: 7 or 10.

6. The method according to claim 4, wherein the antibody is a monoclonal antibody.

7. The method according to claim 6, wherein the monoclonal antibody is Golimumab, Infliximab or Adalimumab.

8. The method according to claim 1, wherein the TNF antagonist comprises a fragment of TNF and/or TNFR.

9. The method according to claim 8, wherein the TNF antagonist is Etanercept.

10. The method according to claim 1, wherein the TNF antagonist decreases the expression of TNF and/or TNFR.

11. The method according to claim 10, wherein the TNF antagonist comprises:
(a) an oligonucleotide or polynucleotide which specifically hybridises to a part of SEQ ID NO: 2, SEQ ID NOs: 6 or 9, or any allelic variant thereof or (b) an oligonucleotide which comprises about 50 or fewer consecutive nucleotides from SEQ ID NO: 1, SEQ ID NOs: 5 or 8 or a variant thereof which has at least about 90% identity to SEQ ID NO: 1, SEQ ID NOs: 5 or 8 over its entire sequence.

12. The method according to claim 1, wherein the LSD is any of Niemann-Pick type C (NPC1), NPC2, Smith-Lemli-Opitz Syndrome (SLOS), an inborn error of cholesterol synthesis, Tangier disease, Pelizaeus-Merzbacher disease, the Neuronal Ceroid Lipofuscinoses, primary glycosphingolipidoses (i.e. Gaucher, Fabry, GM1, GM2 gangliosidoses, Krabbe and MLD), Farber disease and Multiple Sulphatase Deficiency.

13. The method according to claim 1, wherein the method further comprises administering another therapy or agent intended to prevent or treat the LSD.

14. (canceled)

15. An agent for the prevention or treatment of a LSD, comprising a TNF antagonist as an active ingredient.

16. A kit for preventing or treating a LSD, comprising a means for diagnosing or prognosing a LSD and a TNF antagonist.

17. A kit according to claim 16, wherein the means is LysoTracker®.
